(19) United States
(12) Patent Application Publication
(10) Pub. No.: US 2007/0134330 A1
(43) Pub. Date: Jun. 14, 2007

(54) Methods and compositions for improved image quality

(76) Inventors: Marcos A. Barreto, Aguadilla, PR (US); Michael L. Ramirez, Aguadilla, PR (US); Vanessa I. Chinea, Aguadilla, PR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/297,234

(22) Filed: Dec. 8, 2005

(51) Int. Cl. A61K 31/717; A61K 9/24

(52) U.S. Cl. 424/472; 514/57

(57) ABSTRACT

The present invention is directed to consumable coating compositions for use on consumable products, such as pharmaceutical products (e.g., tablets, pills, capsule, gel cap, caplet).

### FIELD OF THE INVENTION

The present invention generally relates to inkjet printing, and in particular, to coatings and methods suitable for use on consumable products.

### BACKGROUND OF THE INVENTION

Consumable products such as pharmaceutical tablets, pills, and capsules, usually include a number of markings. The marks, including printed information, typically include indicia such as logos, names, numbers, letters, bar codes, dosage; that may be used to provide information such as the product, source, and expiration date. Such information is also useful in guiding the dispensing and administration of the product to patients.

Traditional methods of marking pharmaceutical products include coloring the pharmaceutical products with FDA certified colorants, altering the surface appearance of the pharmaceutical products through engravings, applying a label to the surface of the pharmaceutical products, or painting the pharmaceutical product. Examples of such methods include rotogravure, flexographic, and pad printing.

While these methods and formulations are somewhat effective in marking or otherwise distinguishing pharmaceutical product, they necessitate contact with the pharmaceutical. Any such contact with the pharmaceutical products increases the likelihood of causing physical or chemical damage to the pharmaceutical product.

It would be desirable to have methods and formulations for coating consumable products with improved surfaces.

### SUMMARY OF THE INVENTION

The present invention is directed to biocomapatible coating compositions for use on biocompatible products, such as pharmaceutical products, foodstuff, and food related products. In an embodiment, the coating composition is a consumable coating composition formulated by combining an aqueous vehicle and at least one consumable polymer. In an embodiment, the aqueous vehicle comprises at least one consumable water-soluble organic solvent. In an embodiment, the consumable product has a surface with an identifying marking thereon. In an embodiment, the marking is disposed on the surface using an inkjet device.

### BRIEF DESCRIPTION OF DRAWINGS

FIG. 1 is an illustration of a consumable product embodying features of the present invention.

FIG. 2A through 5B are representative images of biocompatible products exhibiting different image quality.

FIG. 6 is a flow chart illustrating a method for forming a biocompatible coating on a biocompatible product, according to an exemplary embodiment.

FIG. 7 is a flow chart illustrating a method for forming a marking on a coated biocompatible product, according to an exemplary embodiment

### DETAILED DESCRIPTION OF EMBODIMENTS OF THE INVENTION

The present invention is directed to biocompatible coating compositions for use on biocompatible products, including consumable products such as pharmaceutical products (e.g., tablets, pills, capsule, gel cap, caplet), foodstuff (e.g., fruit peel) and food related products (e.g., egg shells); as well as healthcare product (e.g., medical devices, medical supplies, or “direct human contact” products such as pacemakers, gauzes, band aids, dental products etc.), and wellness products (e.g. cosmetics). In an embodiment, the biocompatible coating composition is a consumable coating composition.

Coatings embodying features of the present invention, provide improved surfaces for printing of markings or indicia on biocompatible products. The present coatings enable printing of markings or indicia having high print quality. In an embodiment, coatings embodying features of the present invention enable printing with aqueous inks, such as aqueous inks formulated for use with inkjet printing systems. In an embodiment, the marking is disposed on the surface using an inkjet device.

The coatings may be applied to all or a discrete area on the surface of the biocompatible product. Typically such discrete area has a surface area at least as large as that of the image being printed thereon. The coating may be applied on the biocompatible product in a number of ways including contact and non-contact methods such as: mechanical (e.g., swab or brush), atomization (e.g., inkjet such as thermal or piezo inkjet, sprayer), direct polymer application, total or partial immersion of product into coating composition, deposit of macro drops, or traditional coating application methods as fluid-bed process or inside a coating pan, tumble, etc.

In an embodiment, a mask may be used to more precisely define the area to be covered by the coating, as for example, when the coating is applied using spray techniques.

In an embodiment, the coating is formed by application of a biocompatible (e.g., consumable) aqueous solution or mixture including a biocompatible (e.g., consumable) polymer which is water soluble or water swellable. In an embodiment, the aqueous solvent further includes at least one water-soluble organic solvent such as an alcohol (e.g., ethanol, n-propanol, n-butanol). In an embodiment, the polymer is dissolved or dispersed in the aqueous solvent including the at least one water-soluble organic solvent.

Consistent with the invention, various types of additives such as plasticizers, may be employed in the formulations used for providing such coatings; such additives configured to enhance or optimize the properties of the coating formulation and the final coating. The components of the coating formulation may be selected from the list of compounds found in, but not limited to, the Generally Regarded as Safe (“GRAS”) list sponsored by the U.S. Food and Drug Administration (“FDA”).

In addition, biocompatible products having coatings according to the present invention, provide for being marked using a wider range of printing techniques, in particular water-based inks such as inkjet inks. The use of inkjet systems to print on biocompatible products having coatings according to the present invention, provide for easier selection, control, alterations, and design change as the image is stored as data and not fixed on a contact number.

As used in the present specification and the appended claims, the term “biocompatible” is meant to be understood as any composition, including consumable coatings, which is biologically compatible with a human (or other mammals) by not producing a toxic, injurious, or adverse immunological response in living tissue. The term “edible” or “consumable” is meant to be understood broadly as any composition that is suitable for human (or mammals) consumption (including compositions taken or placed intracorporeally such as dental products). Similarly, the phrases “biocompatible product,” biocompatible coating, “biocompatible ink;” “edible product,” “edible coating,” “edible ink;” “consumable product,” “consumable coating,” amd “consumable ink;” are meant to be understood as any product, coating, or ink, that is biocompatible with human (or other mammals), and/or suitable for human (or other mammals) consumption, and complies with applicable standards such as food, drug, and cosmetic (FD&C) regulations in the United States, Eurocontrol experimental centre (E.E.C.) standards in the European Union or other similar regulatory agencies. For purposes of description of the invention, hereinafter consumable products such as pharmaceutical products may be used to describe the features of the invention. Similarly, consumable ingredients and coatings may be used to describe the coating composition and its components. It should be understood by those skilled in the art that such usage is not intended to limit the scope of the invention and its applicability to biocompatible products and coatings.

As used herein, wt. % or % (w/w), represents mass of the solute (or ingredient whether liquid, solid, or gas) for every 100 parts of the mass of the final solution or mixture. By way of example, a mixture of 5% of polymer in water, represents a mixture made by adding 5 grams of polymer and 95 grams of water to make up to 100 grams of total mixture of the formulation.

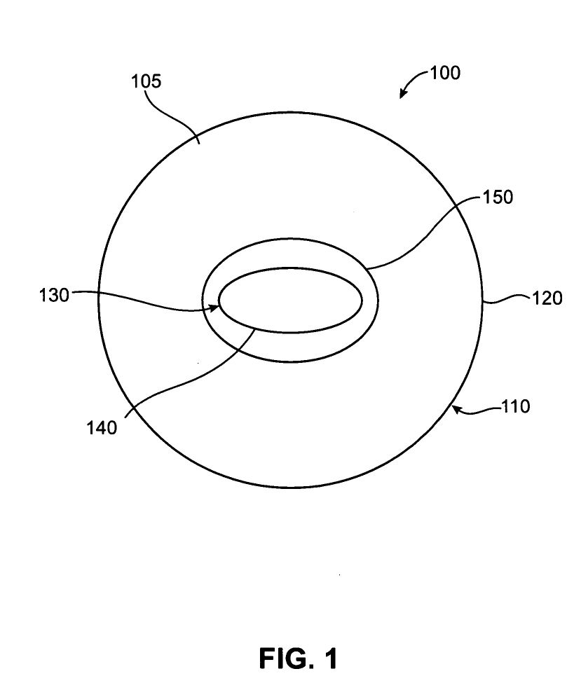

Now referring to FIG. 1, an exemplary marked consumable product 100 having a surface 105, such as pharmaceutical product 110 embodying features of the present invention is shown. In the embodiment shown, the pharmaceutical product 110 is a pill 120, having an indicia (or marking) 130, such as product name 140, printed thereon. As shown, the product name 140 or other indicia or marking, compromises the outward appearance of the pharmaceutical product 110. In the embodiment shown, the marking or indicia 130 is printed on a coated surface 150 of the pharmaceutical product 110. As can be appreciated by those skilled in the art, the coated surface 150 may cover all or a portion of the pharmaceutical product 110. Typically such coated surface has a surface area at least as large as that of the marking being printed thereon.

The coated surface 150 is formed by the application of disposing of a consumable coating formulation comprising an aqueous vehicle and a consumable polymer. In an embodiment, the aqueous vehicle comprises at least one water-soluble organic solvent, such as ethanol, n-propanol, n-butanol, or combinations thereof.

Consistent with this invention, various types of additives, may be employed in the coating formulation configured to enhance or optimize the properties of the coating formulations and/or to optimize or improve the manufacturing process. Examples of such additives include surfactants, buffers, humectants, preservatives, anti-foaming agents, sweeteners, flavoring agents, colorants, and viscosity modifiers. The components of the coating formulation may be selected from the list of compounds found in, but not limited to, the Generally Regarded as Safe (“GRAS”) list sponsored by the U.S. Food and Drug Administration (“FDA”).

In an embodiment, the at least one polymer is a consumable polymer, having an average molecular weight ranging from about 4,000 to about 2,000,000 g/mol, from about 10,000 to about 200,000 g/mol, or from about 10,000 to about 70,000 g/mol. In an embodiment the consumable polymer is a cellulose ether polymer. Cellulosic polymers are made from a naturally occurring polymer cellulose that is obtained from wood pulp and/or cotton fibers. Cellulose can be converted to useful derivates by etherification. Cellulose is chemically modified to produce cellulose ether polymers, such as Carboxymethylcellulose (CMC), Ethylhydroxyethylcellulose, Hydroxyethylcellulose, Hydroxypropylcellulose, Methylhydroxyethylcellulose and Methylhydroxypropylcellulose Methyl cellulose (MC), Methylhydroxyethyl cellulose (MHEC or HEMC), or Methylhydroxypropyl cellulose (MHPC or HPMC).

The amount of polymer (based on weight and calculated based on the total weight of the coating formulation) added to make up the coating formulation ranges generally from about 0.5 to about 15.0%, from about 1% to about 7%, or from about 1% to about 3%. Representative polymers for use in the coating formulation include, but are not limited to: cellulose ethers such as MC (methyl cellulose), EC (ethyl cellulose), HPC (hydropropyl cellulose), HEC (hydroxyethyl cellulose), HPMC (hydroxypropylmethyl cellulose), hydroxyethylmethyl cellulose, sodium carboxymethylcellulose; vinyl Polymers such as polymethacrylates, PVA (polyvinyl alcohols), PVP (polyvinylpyrrolidone), poly(acrylic acid), PVME (polyvinylmethyl ethers); polyesters such poly(lactide) and related copolymers, poly(e-caprolactone); silicones; polymethacrylates such as poly (butyl methacrylate,(2-dimethyl aminoethyl)methylacrylate), poly (methacryllic acid, methacrylate); polysaccharides, resins, gums, and related polymers such as pullulan, pectin, chitosan, cartageenan, tragacanth, acacia, poly(alginic acid), xanthan gum, cellulose gum, guar gum, gum arabic, shellac, gluten; natural or chemically modified starches such as those from potato, wheat, corn, rice tapioca, corn protein (zein); and other polymers such as gelatin, polyanhydrides, polyethylene glycols, polyethylene oxides, polyamides, polioxazolidones, and polyoxazolines (poly(2-ethyl 2-oxazoline)); and combinations thereof.

The coating formulation vehicle facilitates dispersion or dissolution of the various ingredients of the coating formulation while enabling selectively controlled transport of the polymer and other ingredients to the consumable product 100, such as the pharmaceutical product 110. In an embodiment, the coating formulation vehicle may also act as a binder to affix the polymer to the surface of the consumable product 100. In an embodiment, the coating formulation vehicle is an aqueous-based vehicle. According to an embodiment, the aqueous coating vehicle includes water, and at least one water-soluble organic solvent.

The selection of a suitable coating formulation depends on requirements of the specific application, such as the desired physicochemical properties of the formulation such as surface tension and viscosity, the polymer type, performance properties such as drying time of the coating formulation, substrate compatibility and the type of substrate (e.g., the pharmaceutical product composition and/or other surface coating as it affects print quality of the marking 130), onto which the coating formulation is applied, as well as the nature of the printing liquid such as the inkjet ink which is used to print the marking 130. It should be appreciated that the various ingredients may have an effect on more than one property of the consumable coating formulation.

The composition of the vehicle component of the consumable formulation may comprise 100% water, from about 1 to about 100% water, from about 20 to about 95% water, or from about 25 to about 80% water, based on the total weight of the aqueous vehicle (or carrier medium). The water-soluble organic solvent(s), when present, independently, may be added to the coating formulation in an amount generally ranging from about 0 to about 99 wt % of the total weight of the coating formulation; from about 20% to about 75 wt %, or from about 65 to about 70 wt %. The water-soluble organic solvent, may affect one or more of the properties of the coating formulation including dry time, surface tension, viscosity, as well as acting as a humectant.

In an embodiment, the water-soluble organic solvent is a mono or poly alcohol. In an embodiment the water-soluble organic solvent is a mono alcohol including, linear or branched C1-C6 alcohols, such as ethyl alcohol, n-propanol, n-butnaol, 2-propanol, and combinations thereof. Other representative water-soluble organic solvents, include, but are not limited to: sorbitol, propylene glycol, glycerine, polyethylene glycol (“PEG”) 200-8000, normally PEG 400, mannitol, xylitol, hexylene glycol, SUP water; and mixtures of thereof.

The amount of pH controlling additive (based on weight and calculated based on the total weight of the coating formulation) added to make up the coating formulation ranges generally from about 0 to about 15.0%, typically from about 0.1% to about 5%. Representative additives for controlling the pH of the coating formulation include, but are not limited to: sodium dihydrogen phosphate (NaH₂PO₄), potassium phosphate monobasic, 4-Morpholinepropanesulfonic acid; 3-(N-Morpholino)propanesulfonic acid; n-(3-sulfopropyl morpholine (“MOPS”), tris-hydroxymethyl aminomethane (“Tris”), ammonium acetate, acetic acid, hydrochloric acid, sulfuric acid, phosphoric acid, ammonium hydroxide, sodium hydroxide, and mixture thereof.

The amount of surfactant (based on weight and calculated based on the total weight of the coating formulation) added to make up the coating formulation ranges generally from about 0 to about 10%, typically from about 0.1% to about 3%. Representative additives for controlling the surface tension (“surfactant”) of the coating formulation include, but are not limited to: polysorbate 20, polysorbate 40, polysorbate 60, polysorbate 80, pluronic F-127, sodium lauryl sulfate, sorbitan monopalmitate, sorbitan monostearate, sorbitan trioleate, sorbitan tristearate, lecithin, docusate sodium, glyceryl monooleate, poloxamer (polyethylene-propylene glycol copolymer), hydrolyzed polyvinyl alcohol, 2-propanol, ethyl alcohol, N-butyl alcohol, and mixture thereof

The viscosity modifier additive (based by weight and calculated based on the total weight of the coating formulation) added to make up the coating formulation ranges generally from about 0 to about 10.0%, typically from about 0.1% to about 2%. Representative additives for controlling the viscosity of the coating formulation, include, but are not limited to: sodium alginate and other algin derivatives, polyvinyl pyrrolidininone (e.g., molecular weight of about 360000 Da), propylene glycol alginate, hydroxypropylmethylcellulose (e.g., hypromellose 2910), pullulan, polyvinyl alcohol (e.g., molecular weight of about 30000 to about 70000 Da), carrageenan, gelatin, ethylcellulose, guam guar, xanthan gum, hydroxyethyl cellulose, hydroxypropyl cellulose, maltodextrin, corn syrup solids, and combinations thereof.

The antioxidant additive (based on weight and calculated based on the total weight of the coating formulation) added to make up the coating formulation ranges generally from about 0 to about 5.0%, typically from about 0.1% to about 1%. Representative antioxidant additives, include, but are not limited to: sodium ascorbate, sodium thiosulfate, butylated hydroxytoluene (BHT), butylated hydroxyanisole (BHA), monothioglycerol, fumaric Acid, DL-Malic Acid, tocopheroles, citric acid, propyl gailate, and combinations thereof.

The preservative additive (based on weight and calculated based on the total weight of the coating formulation) added to make up the coating formulation ranges generally from about 0 to about 50%, typically from about 0.5% to about 10%. Representative preservatives (or biocide) for controlling microbial growth in the coating formulation include, but are not limited to: sodium benzoate, potassium sorbate, methylparaben, propylparaben, ethylparaben, sodium lactate, benzyl alcohol, 2-propanol, ethyl alcohol, n-butyl alcohol, potassium metabisulfite, propionic acid, succinic acid, sodium propionate, sorbic acid, and combinations thereof.

The anti-foaming additive (based on weight and calculated based on the total weight of the coating formulation) added to make up the coating formulation ranges generally from about 0 to about 20%, typically from about 0.1% to about 5%. Representative anti-foaming additives, and the exemplary amounts thereof, based by weight, based on the total weight of the coating formulation, added to make up the inkjet coating formulation, include, but are not limited to: simethicone (typically at 0.001%), dimethicone (typically at 0001%), n-butyl alcohol, ethanol, isopropyl alcohol, oleyl alcohol, and combinations thereof.

The flavoring (or flavor enhancing) agent additive (based on weight and calculated based on the total weight of the coating formulation) added to make up the coating formulation ranges generally from about 0 to about 10%, typically from about 0.1% to about 5%. Representative flavoring agents, include, but are not limited to: trans-anethole, benzaldehyde, n-butyl alcohol, n-butylamine, ethyl maltol, sucrose, dextrose, fructose (e.g., D-fructose), lactose, polydextrose, sorbitol, xylitol, saccharin, aspartame, acesulfame-K, maltol, vanillin, natural fruit flavors (e.g., lemon, orange), garlic, herbal spices, hydrolyzed vegetable protein, and combinations thereof.

The vitamin and minerals to improve the nutritive value of foods, (based on weight and calculated based on the total weight of the coating formulation) added to make up the coating formulation ranges generally from about 0 to about 10%, typically from about 0.1% to about 5%. Representative vitamin and minerals, include, but are not limited to: potassium iodide (iodide), vitamin D, thiamine mononitrate (vitamin B1), ribiflamin (vitamin B2), ascorbic acid (vitamin C), niacin, vitamin A palmitate, ferrous sulfate (iron).

The colorant may be a dye, a pigment, or a combination of both. More than one dye or pigment may be used in the coating formulation. The colorant, (based on weight and calculated based on the total weight of the coating formulation) added to make up the coating formulation ranges from about 0 to about 20%, generally, from about 0.1 to about 8%, typically from about 1 to about 7%, normally from about 0.5 to about 5%.

Representative colorants include, but are not limited to: isoprenoid derivatives such as carotenoids, and xanthophylls; tetrapyrrole derivatives such as chlorophylls, porphyrin, heme pigments, and bilins; benzopyran derivatives such as anthocyanins, flavones, flavonoids, tannis (e.g., catechin, theaflavin); betalain; phenalone; anthroquinone; FD&C colorants such as allura Red (FD&C Red 40), sunset yellow (FD&C Yellow 6), acid yellow 23 (FD&C Yellow 5), erioglaucine disodium salt (FD&C Blue 1), indigo carmine (FD&C Blue 2), fast green FCF; brilliant blue FCF, riboflavin 5′-phosphate; erythrosine (FD&C Red 3); phloxine B; betalins, anthocyanins, caramels; carmine 40; beet power, curcumin; annatto; eosin Y; fluorescein dyes; and combinations thereof.

Exemplary composition, methods of making, and use thereof, embodying features of the present invention are described below.

### EXAMPLES

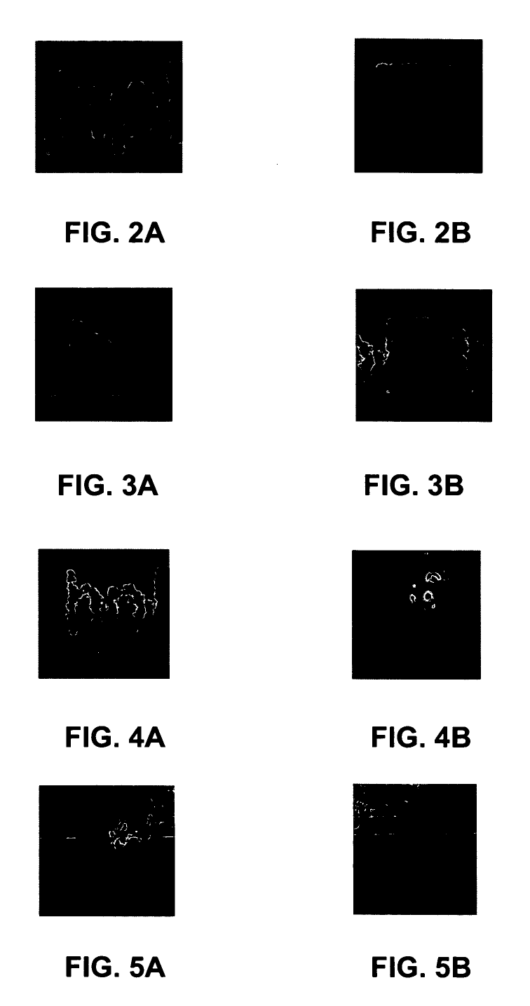

Coating formulations were made and applied onto a range of biocompatible products such as pharmaceutical tablets, confectionaries, egg shell, chewing gum, and gauze (e.g., surgical gauze). A range of coating techniques such as those described above (e.g., inkjet, spray, swabbing, macro drop) were utilized in the application of the coatings. Different performance attributes of the formulated coatings were observed and/or measured in an effort to assess the benefits attained in the practice of the invention, such as, but not limited to, print quality (e.g., edge-acuity of the printed image, uniformity of the printed image, and color density) and printability with inkjet dispensers. The summary of exemplary composition of the coating formulations and their respective overall print quality performance is shown in Table I. Exemplary images representing the visual scale are shown in FIGS. 2A through 5B. Overall image quality performance was ranked on a scale of 1-4, with 1 representing the best image quality. By way of example, scores of 1 and 4; respectively; exhibited excellent area fill uniformity (homogeneous appearance of pattern with no streaked or banded pattern) and excellent area fill uniformity (homogeneous appearance of pattern with no streaked or banded pattern); and very poor edge-acuity in the letters (many distortions of characters' edge) and very poor area fill uniformity (a very non-homogeneous appearance of solid area with very streaked or banded pattern).

TABLE I

| | | Overall Print |
|---|---|---|
| Ingredient | Weight % | Quality Rating |
| Control | | |
| Control Samples (no coating according | | 4 |
| to the present invention) | | |
| EXAMPLE 1 | | |
| HPMC (hydroxypropylmethyl | 2.0% | 1 |
| cellulose) | | |
| Water | 29.1% | |
| Ethyl alcohol | 68.9% | |
| EXAMPLE 2 | | |
| HPMC (hydroxypropylmethyl | 1.2% | 1 |
| cellulose) | | |
| Water | 29.3% | |
| Ethyl alcohol | 69.5% | |
| EXAMPLE 3 | | |
| Poly(2-ethyl 2-oxazoline) | 5.9% | (1-3) avg. 2 |
| Water | 28.0% | |
| Ethyl alcohol | 66.1% | |
| EXAMPLE 4 | | |
| PVP (Polyvinylpyrrolidone) | 7.1% | (1-4) avg. 2.5 |
| Water | 27.5% | |
| Ethyl alcohol | 65.4% | |
| EXAMPLE 5 | | |
| PVP (Polyvinylpyrrolidone) | 1.2% | 1 |
| Water | 29.4% | |
| Ethyl alcohol | 69.4% | |
| EXAMPLE 6 | | |
| PVP (Polyvinylpyrrolidone) | 0.5% | (1-3) avg. 2 |
| Water | 78.8% | |
| Ethyl alcohol | 20.7% | |
| EXAMPLE 7 | | |
| Pullulan | 5.0% | 1 |
| Water | 95% | |
| Ethyl alcohol | 0% | |
| EXAMPLE 8 | | |
| Pullulan | 2.2% | (1-4) avg. 2.5 |
| Water | 54.7% | |
| Ethyl alcohol | 43.1% | |

As can be seen from the data in Table I and FIGS. 2A through 5B, samples prepared according to the present invention provided the best overall image performance.

In an embodiment, indicia (or marking) 130 is produced by applying an inkjet ink formulation, according to the present invention, comprising an aqueous vehicle; at least one colorant; such as dyes or pigments, or a combination thereof; and at least one water-soluble organic solvent, such as ethanol, n-propanol, n-butanol, or combinations thereof. Consistent with the invention, various types of additives, may be employed in the inks configured to enhance or optimize the properties of the ink compositions (or the overall printing system). Examples of such additives include surfactants, buffers, humectants, preservatives, anti-foaming agents, chelating agents (or sequestering agents), flavoring agents, and viscosity modifiers. The components of the ink may be selected from the list of compounds found in, but not limited to, the Generally Regarded as Safe (“GRAS”) list sponsored by the U.S. Food and Drug Administration (“FDA”).

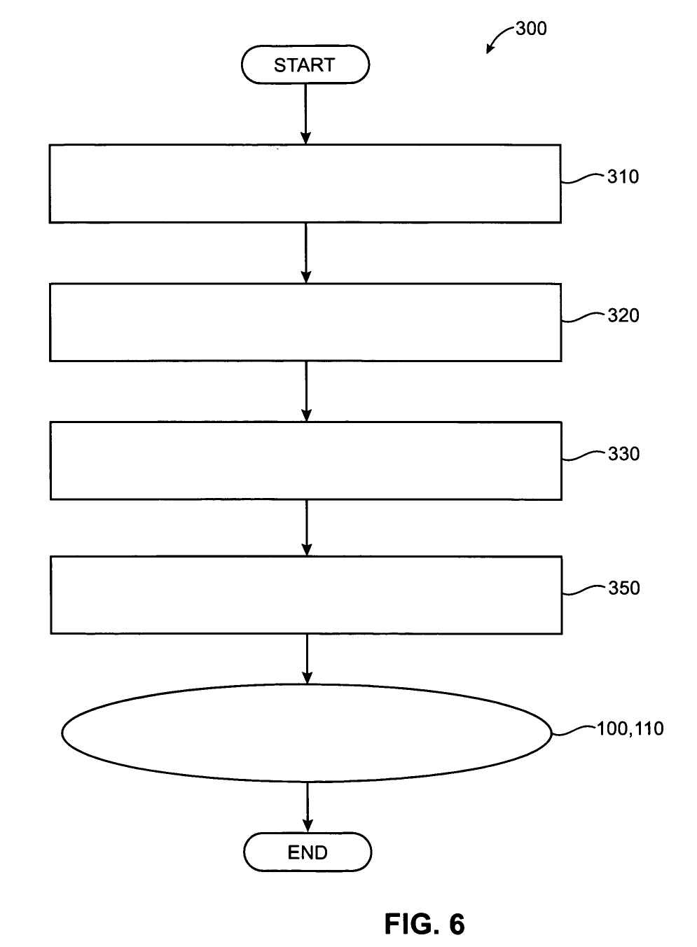

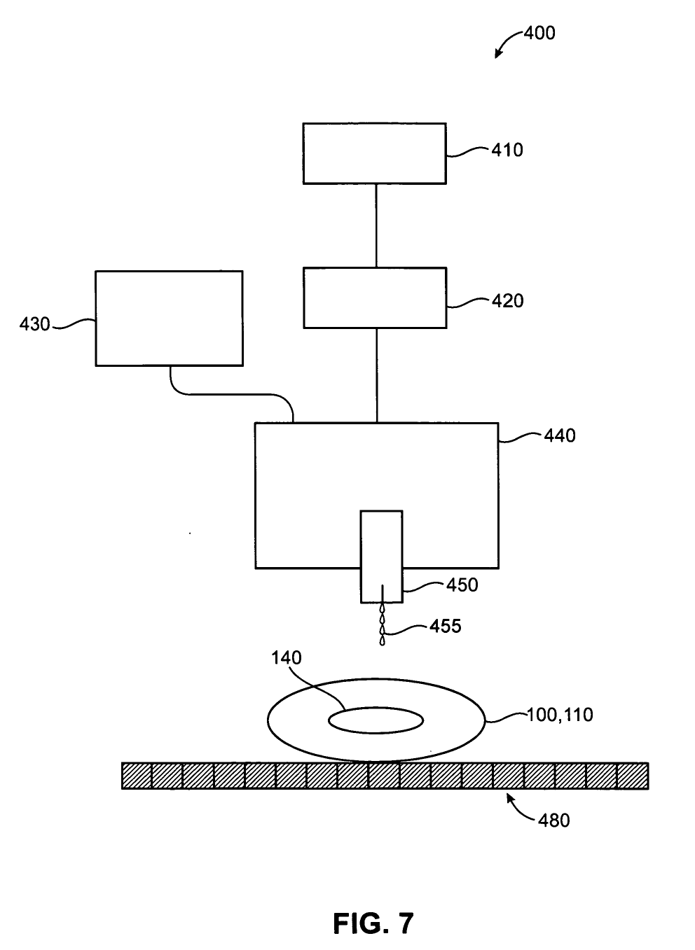

The coating formulation may be made, by way of an exemplary method, by combining a water, organic solvent, polymer, additive, and other ingredients. Preferably, the vehicle (e.g., in an embodiment, water and the organic solvent) and other additives are first combined forming a solution followed by addition of the polymer. As shown, in FIG. 6, the coating formulation is made according to an exemplary process, by first, formulating an exemplary coating formulation vehicle including water and a water-soluble organic solvent (step 310). Once the coating formulation vehicle is formulated, other additives may be added to the vehicle (step 320). The vehicle and other additives are mixed and the polymer is added (step 330), resulting in coating formulation 350. It should be appreciated that present order of the steps may be changed (e.g., additives added before addition of the polymer) to suit the process (e.g., equipment) and the ingredients of the coating formulation. The coating formulation 350 is then applied by way of any of the earlier mentioned methods onto the consumable product 100, such as pharmaceutical product 110. The product with the coating applied thereon is then let dry, by suitable methods (e.g., air dry), to provide an unmarked product 105 with the coated surface 155 thereon. Thereafter, the unmarked product 100 may receive the marking or indicia 140 thereon according to the exemplary method described below regarding FIG. 7.

FIG. 7, illustrates an exemplary system 400 usable in creating markings or indicia 140, on the consumable product 100 (or unmarked product 105) such as pharmaceutical product 110, using ink compositions 455 as described earlier and those described in co-pending U.S. patent application Ser. No. 10/703,697, entitled System and Method for An Edible, Optically Invisible Ink, filed Nov. 6, 2003; and Ser. No. ______ (attorney docket no. 200505577-1), entitled Consumable Inks with Improved Image Performance, filed concurrently herewith; both of which are assigned to the same assignee as the present invention.

As shown in FIG. 7, an exemplary system 400 for marking product 100, 105, 110, includes a computing device 410 controllably coupled through a servo mechanism 420 to a moveable carriage 440 having an ink-jet dispenser 450 (e.g., printhead) disposed thereon. A material reservoir 430 is also coupled to the moveable carriage 440, and consequently to the inkjet dispenser 450. A support surface 480 is located adjacent to the inkjet dispenser 450 having a pharmaceutical product 110 disposed thereon. While the present embodiment is described in the context of marking a pharmaceutical product 110 with an edible ink 455; the present system and method may be used to mark any number of biocompatible products, such as, but not limited to, consumable products, food products, dental products, healthcare products, wellness products.

The computing device 410 controls the selective deposition of the ink 455 on the pharmaceutical product 110. A representation of a desired image or label may be formed using a program hosted by the computing device 410. That representation may then be converted into servo instructions that are then housed in a processor readable medium (not shown). When accessed by the computing device 410, the instructions housed in the processor readable medium may be used to control the servo mechanisms 420 as well as the movable carriage 440 and ink-jet dispenser 450. The computing device 410 illustrated in FIG. 7 may be, but is not limited to, a workstation, a personal computer, a laptop, a personal digital assistant (“PDA”), or any other processor containing device.

The moveable carriage 440, as shown, is a moveable material dispenser that may include any number of inkjet material dispensers 450 configured to dispense the edible ink 455. The moveable carriage 440 may be controlled by the computing device 410 and may be controllably moved by, for example, a shaft system, a belt system, a chain system, etc. making up the servo mechanism 420. As the moveable carriage 440 operates, the computing device 410 may inform a user of operating conditions as well as provide the user with a user interface.

As the image or indicia 140 is printed on the pharmaceutical product 105, 110, the computing device 410 may controllably position the moveable carriage 440 and direct one or more of the inkjet dispensers 450 to selectively dispense the ink 455 at predetermined locations on the pharmaceutical product 110 as digitally addressed drops, thereby forming the desired image on the coated surface 150. The inkjet material dispensers 450 may be any type of inkjet dispenser configured to perform the present method including, but in no way limited to, thermally actuated inkjet dispensers, mechanically actuated inkjet dispensers, electrostatically actuated ink-jet dispensers, magnetically actuated dispensers, piezoelectrically actuated dispensers, continuous inkjet dispensers, etc. Additionally, the ink-jet material dispenser 450 can be heated to assist in dispensing the ink 455.

The material reservoir 430, fluidly coupled to the ink-jet material dispenser 450, houses the ink 455 prior to printing. The material reservoir 430 may be any sterilizeable container configured to hermetically seal the ink 455 prior to printing and may be constructed of any number of materials including, but in no way limited to metals, plastics, composites, or ceramics.

As shown, support surface 480 may transport and/or positionally secure a pharmaceutical product 110 during the printing operation.

While particular forms of the invention have been illustrated and described herein, it will be apparent that various modifications and improvements can be made to the invention. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is intended that this invention to be defined by the scope of the appended claims as broadly as the prior art will permit.

What is claimed is:

1. A biocompatible coating composition, comprising:
a. an aqueous vehicle comprising at least one consumable water-soluble organic solvent; and
b. a consumable polymer.

2. A coating composition according to claim 1, wherein the biocompatible coating is consumable.

3. A coating composition according to claim 2, wherein the consumable polymer comprises at least one polymer selected from the group consisting of cellulose ethers, vinyl Polymers, polyesters, silicones, polymethacrylates, polysaccharides, resins, gums, natural or chemically modified starches, gelatin, polyanhydrides, polyglycols, polyoxides, polyamides, polioxazolidones, polyoxazolines, and combinations thereof.

4. A coating composition according to claim 3, wherein the consumable polymer comprises at least one polymer selected from the group consisting of cellulose ethers such as MC (methyl cellulose), EC (ethyl cellulose), HPC (hydropropyl cellulose), HEC (hydroxyethyl cellulose), HPMC (hydroxypropylmethyl cellulose), hydroxyethylmethyl cellulose, sodium carboxymethylcellulose; vinyl Polymers such as polymethacrylates, PVA (polyvinyl alcohols), PVP (polyvinylpyrrolidone), poly(acrylic acid), PVME (polyvinylmethyl ethers); polyesters such poly(lactide) and related copolymers, poly(e-caprolactone); silicones; polymethacrylates such as poly (butyl methacrylate, (2-dimethyl aminoethyl)methylacrylate), poly (methacryllic acid, methacrylate); polysaccharides, resins, gums, and related polymers such as pullulan, pectin, chitosan, cartageenan, tragacanth, acacia, poly(alginic acid), xanthan gum, cellulose gum, guar gum, gum arabic, shellac, gluten; natural or chemically modified starches such as those from potato, wheat, corn, rice tapioca, corn protein (zein); and other polymers such as gelatin, polyanhydrides, polyethylene glycols, polyethylene oxides, polyamides, polioxazolidones, and polyoxazolines (poly(2-ethyl 2-oxazoline)); and combinations thereof.

5. A coating composition according to claim 4, wherein the consumable polymer comprises at least one polymer selected from the group consisting of cellulose ethers.

6. A coating composition according to claim 5, wherein the consumable polymer comprises at least one cellulose ether polymer selected from the group consisting of MC (methyl cellulose), EC (ethyl cellulose), HPC (hydropropyl cellulose), HEC (hydroxyethyl cellulose), HPMC (hydroxypropylmethyl cellulose), hydroxyethylmethyl cellulose, sodium carboxymethylcellulose, and combinations thereof.

7. A coating composition according to claim 6, wherein the cellulose ether consumable polymer comprises hydroxypropylmethyl cellulose.

8. A coating composition according to claim 7, wherein the hydroxypropylmethyl cellulose has a molecular weight ranging from about 4,000 to about 2,000,000 g/mol.

9. A coating composition according to claim 8, wherein the hydroxypropylmethyl cellulose has a molecular weight ranging from about 10,000 to about 200,000 g/mol.

10. A coating composition according to any one of claims 1 through 9, wherein the at least one water-soluble organic solvent includes at least one consumable alcohol.

11. A coating composition according to claim 10, wherein the at least one water-soluble organic solvent includes at least one consumable mono or poly alcohol.

12. A coating composition according to claim 11, wherein the at least one mono alcohol is selected from the group consisting of C1 through C6 alcohols, and combinations thereof.

13. A coating composition according to claim 12, wherein the at least one mono alcohol is selected from the group consisting of ethanol, n-propanol, n-butanol, and combinations thereof.

14. A coating composition according to claim 10, wherein the at least one organic solvent comprises at least one mono and at least one poly alcohol.

15. A coating composition according to claim 14 wherein the at least one poly alcohol is selected from the group consisting of sorbitol, propylene glycol, glycerine, polyethylene glycol (“PEG”) 200-8000, normally PEG 400, mannitol, xylitol, hexylene glycol, SUP water, and combinations thereof.

16. A coating composition according to claim 15, wherein at least one poly alcohol is a polyethylene glycol having a molecular weight ranging from about 200 to about 8000.

17. A coating composition according to claim 15, wherein at least one poly alcohol is a polyethylene glycol having a molecular weight of about 400.

18. A coating composition according to any one of claims 1 through 9, wherein the at least one water-soluble organic solvent includes ethanol and polyethylene glycol 400.

19. A pharmaceutical product, comprising:
a. a surface; and
b. a coating according to any of claims 1 through 18 disposed on at least a portion of the pharmaceutical product surface.

20. A pharmaceutical product according to claim 19, further comprising a marking on at least a portion of the surface.

21. A pharmaceutical product according to claim 20, wherein the marking is disposed on the pharmaceutical product surface by one of a thermally actuated inkjet dispenser, a mechanically actuated inkjet dispenser, an electrostatically actuated ink-jet dispenser, a magnetically actuated dispenser, a piezoelectrically actuated dispenser, or a continuous inkjet dispenser.

22. A method for marking a consumable product comprising:
a. providing a consumable product having a surface;
b. disposing a coating formulation according to any one of claims 1 through 18 on the product surface;
c. disposing a marking on at least a portion of the product surface.

23. A method for marking a consumable product according to claim 22, further comprising depositing the coating formulation to at least a discrete area of the surface of the consumable product using an inkjet device.

24. A method for marking a consumable product according to claim 22, wherein disposing the marking further comprises ink-jetting the marking on at least a portion of the product surface.
